(19) United States
(12) Patent Application Publication
(10) Pub. No.: US 2018/0276337 A1
(43) Pub. Date: Sep. 27, 2018

(54) Method for identifying radiation induced genes and long non-coding RNAs and Application Thereof

(71) Applicant: National Taiwan University, Taipei (TW)

(72) Inventors: Eric Y. Chuang, Taipei City (TW); Tzu-Pin Lu, Taipei City (TW); Mong-Hsun Tsai, Taipei City (TW); Liang-Chuan Lai, Taipei City (TW); Wei-An Wang, Taipei City (TW)

(21) Appl. No.: 15/468,284

(22) Filed: Mar. 24, 2017

(51) Int. Cl. G06F 19/20; C12Q 1/68

(52) U.S. Cl. CPC G06F 19/20; C12Q 2600/158; C12Q 2600/106; C12Q 1/6886

(57) ABSTRACT

The present invention provides a method for identifying radiation induced genes and long non-coding RNAs and its application thereof, the method comprises the steps of: (1). Provide expression values of genes and long non-coding RNAs; (2). Execute weighted gene correlation network analysis (WGCNA) by a computer system to calculate Pearson correlation coefficients of pairs of the genes and long non-coding RNAs based on the expression values of the genes and long non-coding RNAs; and (3). Perform a screening step by the computer system to identify radiation induced genes and long non-coding RNAs based on the Pearson correlation coefficients of the pairs of the genes and long non-coding RNAs with a value more than 0.75.

### TECHNICAL FIELD OF THE INVENTION

The present invention relates to a method for identifying radiation induced genes and long non-coding RNAs (lncRNAs). In particular, some of the radiation induced genes and long non-coding RNAs (lncRNAs) are applied as markers for predicting radiotherapy response of glioblastoma in patients with glioblastoma.

### BACKGROUND OF THE INVENTION

Radiotherapy has become a popular and standard approach for treating cancer patients because it greatly improves patient survival. However, some of the patients receiving radiotherapy suffer from adverse effects and do not obtain survival benefits. This may be attributed to the fact that most radiation treatment plans are designed based on cancer type, without consideration of each individual's radiosensitivity.

Conventional radiotherapy is now combined with images processed by computed tomography to provide a non-invasive and highly tumor-specific treatment plan. Currently, most radiation treatment plans are designed based purely on the cancer type. Challenges arise, however, when individual genetic differences in radiosensitivity lead to different treatment responses among patients receiving the same radiation dose. For example, International Journal of Radiation Oncology* Biology* Physics. 2005; 61(1):196-202 disclosed genetic variants in ATM are associated with differential hypersensitivity to radiation exposure. These results suggest that radiation sensitivity should be taken into consideration when designing the treatment plan.

Based on the aforementioned description, a method for identifying markers for identifying radiation induced markers and then predicting the radiosensitivity of tumor in patients with tumor by the radiation induced markers is required in the near future.

### SUMMARY OF THE INVENTION

In one aspect, the present invention provides a method for identifying radiation induced genes and long non-coding RNAs (lncRNAs). The invention method comprises the steps of (1). Provide expression values of genes and long non-coding RNAs; (2). Execute weighted gene correlation network analysis (WGCNA) by a computer system to calculate Pearson correlation coefficients of pairs of the genes and long non-coding RNAs based on the expression values of the genes and long non-coding RNAs; and (3). Perform a screening step by the computer system to identify radiation induced genes and long non-coding RNAs based on the Pearson correlation coefficients of the pairs of the genes and long non-coding RNAs with a value more than 0.75.

The aforementioned radiation induced genes and long non-coding RNAs are identified from the pairs of the genes and long non-coding RNAs based on the Pearson correlation coefficients of the pairs of the genes and long non-coding RNAs with a value more than 0.75. That is the Pearson correlation coefficients of the pairs of the radiation induced genes and long non-coding RNAs are more than 0.75.

In general, the expression values of genes and long non-coding RNAs are measured in vitro from immortalized B cells (GSE26835) after irradiation by a method comprises microarray.

For developing the method for identifying radiation induced genes and long non-coding RNAs, some tests and algorithm, such as the analysis of variance (ANOVA) test, Tukey's honest significant difference (HSD) test and weighted gene correlation network analysis are performed by a computer system for analyzing the expression values of genes and long non-coding RNAs, and further identify the radiation induced genes and long non-coding RNAs based on the Pearson correlation coefficients of the pairs of the genes and long non-coding RNAs.

Firstly, the analysis of variance (ANOVA) test and Tukey's honest significant difference (HSD) test were performed in microarray dataset of immortalized B cells (GSE26835) to identify differentially expressed genes and lncRNAs (P<0.0001). In total, 1,086 samples were classified into 3 groups, which were 0, 2, and 6 h after irradiation (As shown in FIG. 1). A total of 640 probes including 8 lncRNAs showed significant expression changes between 0 h and 2 h post-irradiation and 1,090 probes including 10 lncRNAs were identified as having expression changes between 0 h and 6 h post-irradiation.

Secondly, a network-based co-expression algorithm, weighted gene correlation network analysis (WGCNA), was performed to identify modules composed of correlated genes and lncRNAs. Comparison of the samples without irradiation (0 h) to the identified set of differentially expressed probes (N=1,244) via WGCNA revealed 2 and 10 modules at 2 h and 6 h post-irradiation, respectively. Among the 12 identified modules, highly correlated interactions between genes and lncRNAs were selected based on their Pearson correlation coefficients (r>0.75). Consequently, a total of 43 interaction pairs triggered by irradiation were identified (As listed in Table 1), which contains 34 genes and 7 lncRNAs. The 34 genes and 7 lncRNAs are determined to the claimed radiation induced genes and lncRNAs.

In another aspect, the present invention provides a method for predicting radiotherapy response of glioblastoma in patients with glioblastoma. The method comprises the steps of: (1). determine expression values of markers in a test sample from the patients with glioblastoma to obtain a test dataset of expression values of the markers, wherein the markers are selected from the group consisting of DDX3Y, EIF1AY, RPS4Y1, USP9Y, RHOC, EPS8L2, ACTA2, MR1, TRAPPC6A, ANXA4, ETHE1, PYCARD, JUP///KRT17, ETFDH, ALDH6A1, RTN1, TP53TG1, LOC100653017 and LOC100506948; (2). compare the test dataset of expression values of the markers to a radiosensitive sample and to a radioresistant sample; (3). classify whether a test dataset of expression values of the markers is significantly within the radiosensitive sample or within the radioresistant sample to assess whether the test sample is radiosensitive (RS) or radioresistant (RR); (4). determine the patients with glioblastoma as in a radiosensitive (RS) group if the test sample is radiosensitive, or determine the patients with glioblastoma as in a radioresistant (RR) group if the test sample is radioresistant; and (5). predict radiotherapy response of glioblastoma in the patients with glioblastoma based on whether the radiosensitive (RS) group or radioresistant (RR) group is treated with radiotherapy.

For developing the aforementioned method, the NCI-60 cell lines, along with the fraction of surviving cells after a radiation dose of 2 Gy (SF2), were utilized as the training set. For the claimed radiation induced genes and long non-coding RNAs, a genetic algorithm (GA) was used to select the best combination of genes and lncRNAs as the markers for developing a model by the support vector machine (SVM) algorithm (As shown in FIG. 1). The GA identified 20 markers, comprising 16 genes and 4 long non-coding RNAs (As listed in Table 2). Lastly, to further confirm that this combination was not identified by random chance, a permutation test was performed by considering various combinations of the claimed radiation induced genes and lncRNAs. The low empirical p-value (0.0014) indicates that the prediction model has a low chance of being identified by chance.

The SVM algorithm is applied to calculate a decision value for determining whether a sample generated from the expression of the 20 markers as listed in Table 2 in NCI-60 cell lines is radiosensitive (RS) or radioresistant (RR). When the decision value is negative, a sample is defined to a radiosensitive sample. In contrast, when the decision value is positive, a sample is defined to a radioresistant sample.

Two microarray datasets from patients with glioblastoma multiforme (GBM) were retrieved, one from The Cancer Genome Altas (TCGA) and another from the Gene Expression Omnibus (GEO) (accession number GSE16011). The datasets were analyzed to evaluate the prediction performance of the developed SVM model. The model was applied to classify the patients into radiosensitive (RS) and radioresistant (RR) groups.

To sum up, the present invention provides the method for identifying radiation induced genes and lncRNAs. In particular, some of the radiation induced genes and lncRNAs are applied as markers for predicting the radiosensitivity of glioblastoma in the patients with glioblastoma. Also the method for predicting radiotherapy response of glioblastoma in patients with glioblastoma is disclosed in the present invention.

### BRIEF DESCRIPTION OF THE DRAWINGS

FIG. 1 is workflow to identify radiation induced genes and lncRNAs, a total of 4 microarray datasets were analyzed. Briefly, differentially expressed genes and lncRNAs were identified in GSE26835. The weighted gene correlation network analysis (WGCNA) method and a genetic algorithm (GA) were performed in to select predictors. A prediction model for radiosensitivity was developed using the support vector machine (SVM) and two external glioblastoma multiforme (GBM) datasets were analyzed;

FIG. 2(a) is the Kaplan-Meier survival curves for the TCGA datasets where the patients received radiotherapy (RT+); FIG. 2(b) is the Kaplan-Meier survival curves for the TCGA datasets where the patients did not receive radiotherapy (RT−); FIG. 2(c) is the Kaplan-Meier survival curves for GSE16011 datasets where the patients received radiotherapy (RT+); and FIG. 2(d) is the Kaplan-Meier survival curves for GSE16011 datasets where the patients did not receive radiotherapy (RT−); and where the patients were classified as radioresistant (RR) and radiosensitive (RS) based on the developed prediction model; and

FIG. 3 is genetic algorithm for feature selection in the prediction model, to ensure that the prediction accuracy value of the model became stable, the selection was repeated for 100 generations.

### BRIEF DESCRIPTION OF THE PREFERRED EMBODIMENTS

In a first embodiment, the present invention discloses a method for identifying radiation induced genes and long non-coding RNAs (lncRNAs). The invention method comprises the steps of (1). Provide expression values of genes and long non-coding RNAs; (2). Execute weighted gene correlation network analysis (WGCNA) by a computer system to calculate Pearson correlation coefficients of pairs of the genes and long non-coding RNAs based on the expression values of the genes and long non-coding RNAs; and (3). Perform a screening step by the computer system to identify the radiation induced genes and long non-coding RNAs based on the Pearson correlation coefficients of the pairs of the genes and long non-coding RNAs with a value more than 0.75.

Accordingly, the invention method for identifying the radiation induced genes and long non-coding RNAs is mainly based on the Pearson correlation coefficients of the pairs of the genes and long non-coding RNAs more than 0.75. That is the radiation induced genes and long non-coding RNAs have the Pearson correlation coefficients of the pairs of the genes and long non-coding RNAs more than 0.75.

In one example of the first embodiment, the expression values of genes and long non-coding RNAs are measured in vitro from immortalized B cells (GSE26835) after irradiation by a method comprises microarray.

In one example of the first embodiment, the radiation induced genes are selected from the group consisting of DDX3Y, EIF1AY, RPS4Y1, USP9Y, KDM5D, TP53I3, RHOC, ASTN2, GAMT, EPS8L2, ACTA2, E2F5, MR1, UTY, TRAPPC6A, FHL2, ANXA4, GLS2, CEACAM1, ETHE1, TSPAN31, PYCARD, CDK2, JUP///KRT17, ATP6V1D, PROCR, ETFDH, ALDH6A1 and RTN1.

In one example of the first embodiment, the radiation induced long non-coding RNAs are selected from the group consisting of TTTY15, TP53TG1, LOC100653079, LOC100653017 and LOC100506948.

For identifying the claimed radiation induced genes and long non-coding RNAs, some tests and algorithm, such as the analysis of variance (ANOVA) test, Tukey's honest significant difference (HSD) test and weighted gene correlation network analysis (WGCNA) are executed by a computer system for analyzing the expression values of genes and long non-coding RNAs, and further identify the radiation induced genes and long non-coding RNAs based on the Pearson correlation coefficients of the pairs of the genes and long non-coding RNAs.

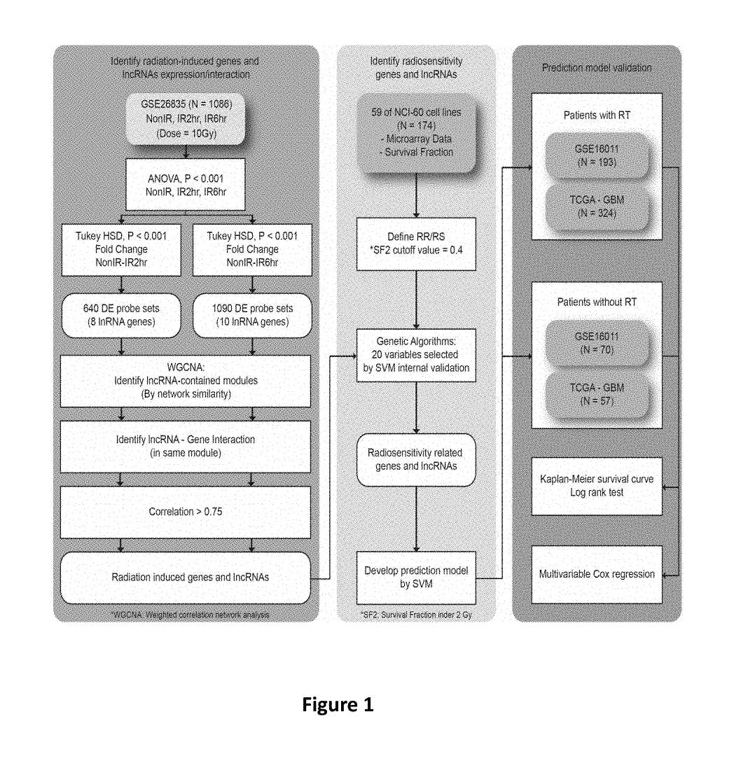

Firstly, the analysis of variance (ANOVA) test and Tukey's honest significant difference (HSD) test were performed in microarray dataset of immortalized B cells (GSE26835) to identify differentially expressed genes and lncRNAs (P<0.0001). In total, 1,086 samples were classified into 3 groups, which were 0, 2, and 6 h after irradiation (As shown in FIG. 1). A total of 640 probes including 8 lncRNAs showed significant expression changes between 0 h and 2 h post-irradiation and 1,090 probes including 10 lncRNAs were identified as having expression changes between 0 h and 6 h post-irradiation.

Secondly, a network-based co-expression algorithm, weighted gene correlation network analysis (WGCNA), was performed to identify modules composed of correlated genes and lncRNAs. Comparison of the samples without irradiation (0 h) to the identified set of differentially expressed probes (N=1,244) via WGCNA revealed 2 and 10 modules at 2 h and 6 h post-irradiation, respectively. Among the 12 identified modules, highly correlated interactions between genes and lncRNAs were selected based on their Pearson correlation coefficients (r>0.75). Consequently, a total of 43 interaction pairs triggered by irradiation were identified as listed in Table 1, which contains 34 genes and 7 lncRNAs. The 34 genes and 7 lncRNAs are defined as the claimed radiation induced genes and lncRNAs.

TABLE 1

| Probe(lncRNA) | Name(lncRNA) | Probe(gene) | Name(gene) | Correlation | Time |
|---|---|---|---|---|---|
| 214983_at | TTTY15 | 205000_at | DDX3Y | 0.935 | 2 h |
| 214983_at | TTTY15 | 204410_at | EIF1AY | 0.921 | 2 h |
| 214983_at | TTTY15 | 201909_at | RPS4Y1 | 0.92 | 2 h |
| 214983_at | TTTY15 | 206624_at | USP9Y | 0.918 | 2 h |
| 214983_at | TTTY15 | 204409_s_at | EIF1AY | 0.907 | 2 h |
| 214983_at | TTTY15 | 206700_s_at | KDM5D | 0.896 | 2 h |
| 214983_at | TTTY15 | 205001_s_at | DDX3Y | 0.892 | 2 h |
| 209917_s_at | TP53TG1 | 210609_s_at | TP53I3 | 0.883 | 2 h |
| 209917_s_at | TP53TG1 | 200885_at | RHOC | 0.847 | 2 h |
| 222271_at | — | 210609_s_at | TP53I3 | 0.839 | 2 h |
| 209917_s_at | TP53TG1 | 215407_s_at | ASTN2 | 0.835 | 2 h |
| 209917_s_at | TP53TG1 | 205354_at | GAMT | 0.823 | 2 h |
| 209917_s_at | TP53TG1 | 218180_s_at | EPS8L2 | 0.82 | 2 h |
| 209917_s_at | TP53TG1 | 200974_at | ACTA2 | 0.814 | 2 h |
| 222051_s_at | — | 221586_s_at | E2F5 | 0.809 | 2 h |
| 222271_at | — | 200974_at | ACTA2 | 0.806 | 2 h |
| 209917_s_at | TP53TG1 | 210224_at | MR1 | 0.801 | 2 h |
| 214983_at | TTTY15 | 211149_at | UTY | 0.798 | 2 h |
| 209917_s_at | TP53TG1 | 207565_s_at | MR1 | 0.797 | 2 h |
| 222271_at | — | 215407_s_at | ASTN2 | 0.796 | 2 h |
| 222271_at | — | 200885_at | RHOC | 0.793 | 2 h |
| 209917_s_at | TP53TG1 | 204985_s_at | TRAPPC6A | 0.793 | 2 h |
| 222271_at | — | 205354_at | GAMT | 0.79 | 2 h |
| 209917_s_at | TP53TG1 | 202949_s_at | FHL2 | 0.789 | 2 h |
| 222271_at | — | 201301_s_at | ANXA4 | 0.783 | 2 h |
| 209917_s_at | TP53TG1 | 205531_s_at | GLS2 | 0.774 | 2 h |
| 209917_s_at | TP53TG1 | 209498_at | CEACAM1 | 0.769 | 2 h |
| 209917_s_at | TP53TG1 | 204034_at | ETHE1 | 0.766 | 2 h |
| 222271_at | — | 203226_s_at | TSPAN31 | 0.765 | 2 h |
| 209917_s_at | TP53TG1 | 221666_s_at | PYCARD | 0.765 | 2 h |
| 222271_at | — | 202949_s_at | FHL2 | 0.764 | 2 h |
| 215708_s_at | LOC100653079 | 211804_s_at | CDK2 | 0.762 | 6 h |
| 222271_at | — | 218180_s_at | EPS8L2 | 0.761 | 2 h |
| 209917_s_at | TP53TG1 | 212236_x_at | JUP /// KRT17 | 0.76 | 2 h |
| 209917_s_at | TP53TG1 | 210223_s_at | MR1 | 0.76 | 2 h |
| 222271_at | — | 210224_at | MR1 | 0.759 | 2 h |
| 222271_at | — | 207566_at | MR1 | 0.759 | 2 h |
| 214657_s_at | LOC100653017 | 208899_x_at | ATP6V1D | 0.757 | 2 h |
| 209917_s_at | TP53TG1 | 203650_at | PROCR | 0.751 | 2 h |
| 214657_s_at | LOC100653017 | 33494_at | ETFDH | 0.75 | 2 h |
| 222271_at | — | 215407_s_at | ASTN2 | 0.75 | 6 h |
| 213447_at | LOC100506948 | 221590_s_at | ALDH6A1 | 0.75 | 2 h |
| 209917_s_at | TP53TG1 | 203485_at | RTN1 | 0.75 | 2 h |

In one preferred example of the first embodiment, the radiation induced genes selected from the group consisting of DDX3Y, EIF1AY, RPS4Y1, USP9Y, RHOC, EPS8L2, ACTA2, MR1, TRAPPC6A, ANXA4, ETHE1, PYCARD, JUP///KRT17, ETFDH, ALDH6A1 and RTN1 are applied as markers for patients with glioblastoma.

In one preferred example of the first embodiment, the radiation induced long non-coding RNAs selected from the group consisting of TP53TG1, LOC100653017 and LOC100506948 are applied as markers for patients with glioblastoma.

In a second embodiment of the present invention, a method for predicting radiotherapy response of glioblastoma in patients with glioblastoma is provided. The method comprises the steps of: (1). determine expression values of markers in a test sample from the patients with glioblastoma to obtain a test dataset of expression values of the markers, wherein the markers are selected from the group consisting of DDX3Y, EIF1AY, RPS4Y1, USP9Y, RHOC, EPS8L2, ACTA2, MR1, TRAPPC6A, ANXA4, ETHE1, PYCARD, JUP///KRT17, ETFDH, ALDH6A1, RTN1, TP53TG1, LOC100653017 and LOC100506948; (2). compare the test dataset of expression values of the markers to a radiosensitive sample and to a radioresistant sample; (3). classify whether a test dataset of expression values of the markers is significantly within the radiosensitive sample or within the radioresistant sample to assess whether the test sample is radiosensitive or radioresistant; (4). determine the patients with glioblastoma as in a radiosensitive (RS) group if the test sample is radiosensitive, or determine the patients with glioblastoma as in a radioresistant (RR) group if the test sample is radioresistant; and (5). predict radiotherapy response of glioblastoma in the patients with glioblastoma based on whether the radiosensitive (RS) group or radioresistant (RR) group is treated with radiotherapy.

For developing the aforementioned method, the NCI-60 cell lines, along with the fraction of surviving cells after a radiation dose of 2 Gy (SF2), were utilized as the training set. For the claimed radiation induced genes and long non-coding RNAs, a genetic algorithm (GA) was used to select the best combination of genes and lncRNAs as the markers for developing a model by the support vector machine (SVM) algorithm (As shown in FIG. 1). The GA identified 20 markers, comprising 16 genes and 4 long non-coding RNAs as listed in Table 2. Lastly, to further confirm that this combination was not identified by random chance, a permutation test was performed by considering various combinations of the claimed radiation induced genes and lncRNAs. The low empirical p-value (0.0014) indicates that the prediction model has a low chance of being identified by chance.

TABLE 2

| Probe Sets | Gene Symbol | Ensembl ID |
|---|---|---|
| 205000_at | DDX3Y | ENSG00000067048 |
| 204410_at | EIF1AY | ENSG00000198692 |
| 201909_at | RPS4Y1 | ENSG00000129824 |
| 206624_at | USP9Y | ENSG00000114374 |
| 200885_at | RHOC | ENSG00000155366 |
| 218180_s_at | EPS8L2 | ENSG00000177106 |
| 200974_at | ACTA2 | ENSG00000107796 |
| 204985_s_at | TRAPPC6A | ENSG00000007255 |
| 201301_s_at | ANXA4 | ENSG00000196975 |
| 204034_at | ETHE1 | ENSG00000105755 |
| 221666_s_at | PYCARD | ENSG00000103490 |
| 212236_x_at | JUP | ENSG00000128422 |
| 210223_s_at | MR1 | ENSG00000153029 |
| 33494_at | ETFDH | ENSG00000171503 |
| 221590_s_at | ALDH6A1 | ENSG00000119711 |
| 203485_at | RTN1 | ENSG00000139970 |
| *209917_s_at | TP53TG1 | ENSG00000182165 |
| *222051_s_at | — | ENSG00000254208 |
| *214657_s_at | LOC100653017 | ENSG00000245532 |
| *213447_at | LOC100506948 | ENSG00000224078 |

The SVM algorithm is applied to calculate a decision value for determining whether a sample generated from the expression of the 20 markers as listed in Table 2 in NCI-60 cell line is radiosensitive (RS) or radioresistant (RR).

The SVM feature weight (w) is firstly calculated by the following function using a computer system with SVM algorithm. The coefficients (coefs) of the 20 markers are listed in TABLE 3

SVM feature weight (w)=t(model$coefs) %*% model SV

TABLE 3

| Probe Sets | Gene Symbol | Coefficient |
|---|---|---|
| 205000_at | DDX3Y | −0.291151742 |
| 204410_at | EIF1AY | 1.128227595 |
| 201909_at | RPS4Y1 | −0.312958353 |
| 206624_at | USP9Y | −0.802155891 |
| 200885_at | RHOC | −0.489540955 |
| 218180_s_at | EPS8L2 | −0.516217949 |
| 200974_at | ACTA2 | 0.462793641 |
| 204985_s_at | TRAPPC6A | 1.75851404 |
| 201301_s_at | ANXA4 | −0.101919582 |
| 204034_at | ETHE1 | −0.35207933 |
| 221666_s_at | PYCARD | −1.071964217 |
| 212236_x_at | JUP | −0.992746799 |
| 210223_s_at | MR1 | 0.406776483 |
| 33494_at | ETFDH | 0.155615068 |
| 221590_s_at | ALDH6A1 | −0.159051022 |
| 203485_at | RTN1 | 0.344587618 |
| 209917_s_at | TP53TG1 | −0.565113311 |
| 222051_s_at | — | −0.067031646 |
| 214657_s_at | LOC100653017 | −0.526942366 |
| 213447_at | LOC100506948 | 1.119826663 |

The decision value is then obtained by the following function and equation using a computer system with SVM algorithm.

Data.scaled=scale(testing_data,model$x.scale[[1]],mode.$x.scale[[2]])

Decision value=t(w %*% t(as.matrix(data.scaled)))−model$rho

When the decision value is negative (decision value <0), the sample is defined to a radiosensitive sample. In contrast, when the decision value is positive (decision value >0), the sample is defined to a radioresistant sample.

Two microarray datasets from patients with glioblastoma multiforme (GBM) were retrieved, one from The Cancer Genome Altas (TCGA) and another from the Gene Expression Omnibus (GEO) (accession number GSE16011). The datasets were analyzed to evaluate the prediction performance of the developed SVM model. The model was applied to classify the patients into radiosensitive (RS) and radioresistant (RR) groups.

In one example of the second embodiment, the method for predicting radiotherapy response of glioblastoma in patients with glioblastoma is applied to a prognosis analysis of the patients with glioblastoma treated with radiotherapy.

In one example of the second embodiment, the expression values of the markers are measured in vitro by a method comprises microarray and real-time polymerase chain reaction (RT-PCR).

In one example of the second embodiment, the test sample comprises lymphocytes sample and peripheral blood sample.

In one example of the second embodiment, the radiosensitive sample and radioresistant sample are determined by a decision value calculated from the expression values of the markers in NCI-60 cell lines by support vector machine, when the decision value is negative, the radiosensitive sample is defined; and when the decision value is positive, the radioresistant sample is defined.

In one example of the second embodiment, the step of classifying whether the test dataset of expression values of the markers is significantly within the radiosensitive sample or within the radioresistant sample comprises use of a predictive algorithm. Preferably, the predictive algorithm is a support vector machine.

In one example of the second embodiment, the steps of (2), (3), (4) and (5) are performed by a computer system.

The following descriptions are to explain both of the method for identifying the claimed radiation induced genes and long non-coding RNAs (lncRNAs) and the method for predicting radiotherapy response of glioblastoma in patients with glioblastoma in more details.

Microarray Datasets

Four datasets analyzed in this study were retrieved from the Gene Expression Omnibus (GEO), CellMiner, and The Cancer Genome Altas (TCGA). The dataset with the largest sample size, GSE26835, was utilized as the identification set to select differentially expressed genes and lncRNAs triggered by irradiation. Subsequently, a prediction model for radiosensitivity was developed based on the expression profiles of the NCI-60 cell lines. Two GBM datasets were used as the validation sets to evaluate the performance of the developed prediction model. All preprocessing procedures and normalization algorithms, including robust multiarray averaging (RMA) and quantile normalization, were performed in R. Although the microarray platforms of these 4 datasets were originally designed to examine gene expression profiles, the studies have shown that a re-analysis of the probe sequences can select the probes targeting lncRNAs. In this study, all those probe sets targeting lncRNAs can be mapped to the Ensembl database.

Identification of Differentially Expressed Genes and lncRNAs Triggered by Irradiation

A protocol to identify the radiation-induced genes and lncRNAs and develop a prediction model is illustrated in FIG. 1. Initially, the ANOVA and Tukey's HSD tests were performed in the samples harvested at 0, 2, and 6 h after radiation exposure in GSE26835 (P<0.0001). To take biological effects into consideration, only probes showing at least 1.3-fold changes were retained for further analyses. Because few published studies have reported the biological functions of lncRNAs, Ingenuity Pathway Analysis (IPA®, QIAGEN Redwood City, www.qiagen.com/ingenuity) was performed only on the differentially expressed genes to characterize their signaling pathways and associated regulators.

Identification of Gene-lncRNA Interaction Pairs

To take the biological functions of lncRNAs into consideration, a network-based co-expression algorithm, WGCNA, was used. The main concept of the WGCNA method is to cluster the genes and lncRNAs with similar expression patterns into a single module based on the hypothesis that genes and lncRNAs involved in the same functional pathway will have highly correlated expression values. All parameters were set as their default values, except that “deepSplit” was used to explore more possible regulations between genes and lncRNAs. Lastly, the Pearson correlation coefficients were calculated for the genes and lncRNAs classified into the same module in order to select the highly correlated gene-lncRNA interaction pairs (r>0.75), and then the claimed radiation induced genes and lncRNAs are identified.

Development of a Prediction Model Using a Genetic Algorithm

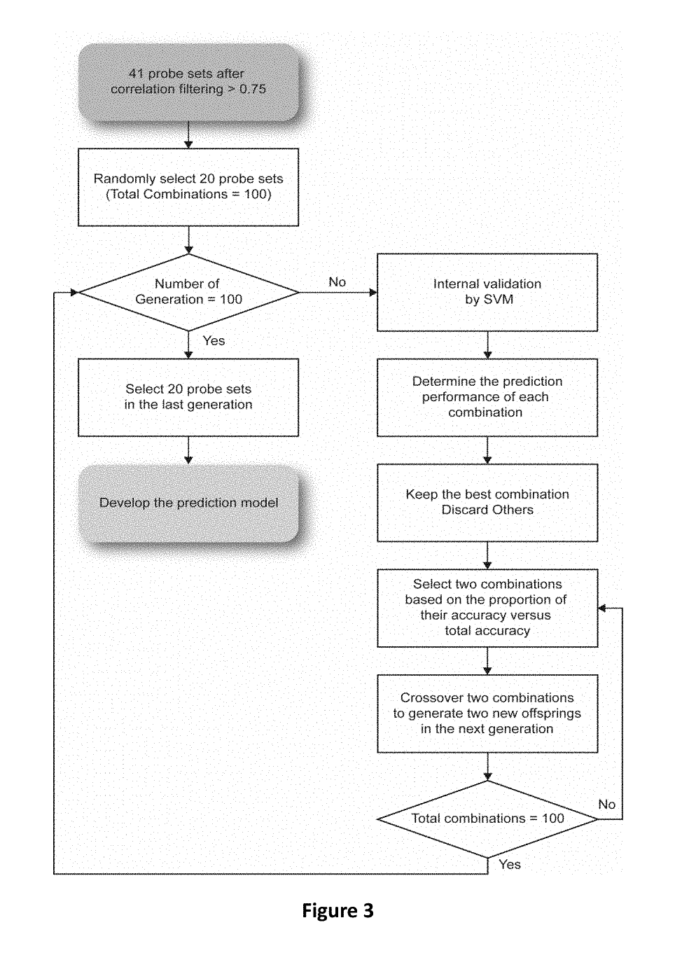

The gene expression profiles and radiation parameters of the NCI-60 cell lines retrieved from CellMiner were analyzed to develop a prediction model. To mimic the situation of radiotherapy in real patients, the survival fraction under 2Gy (SF2) was utilized, and the NCI-60 cell lines were dichotomized into radiosensitive (RS) and radioresistant (RR) groups based on the threshold of 0.4. A genetic algorithm (GA) was designed to select genes and lncRNAs with the highest prediction accuracy in the NCI-60 cell lines (As shown in FIG. 3). In the first generation, we randomly selected 20 predictors from the gene-lncRNA interaction pairs, which included 34 genes and 7 lncRNAs. The random selection of predictors was repeated 100 times to simulate different combinations of genes and lncRNAs. For different combinations of predictors, the SVM algorithm was used to develop prediction models, and their performance was evaluated in the NCI-60 cell lines using 5-fold cross-validation. Subsequently, the combination showing the highest prediction accuracy in the first generation was kept in the second generation. To generate other combinations in the second generation, 2 combinations in the first generation were selected based on the probabilities that were calculated by dividing their accuracy values by the total accuracy values of all combinations. This method is based on the belief that a combination in the first generation with higher accuracy is more likely to be selected in the next generation, which concurs with the concept of “survival of the fittest.” A random exchange of predictors among the 2 selected combinations was performed in order to mimic the process of crossover and to add more diversity to the set of predictors. The procedures to breed a new generation were repeated until 100 generations were simulated, and the prediction model in the last generation produced the highest accuracy for predicting radiosensitivity (SF2). Lastly, a permutation test was performed to evaluate the random chance of identifying 20 markers (predictors) with the same accuracy value. A combination of 20 predictors was randomly selected from the 43 interaction pairs listed in Table 1 and this procedure was repeated 100,000 times to establish a null baseline of prediction accuracy. To evaluate whether lncRNAs raise the prediction accuracy, the comparison of randomly selected predictors included lncRNA probesets. The empirical p-value of a prediction model was determined by comparing its prediction accuracy with the null baseline, that is, by the ranking of the accuracy values.

Validation of Prediction Model in GBM Patients

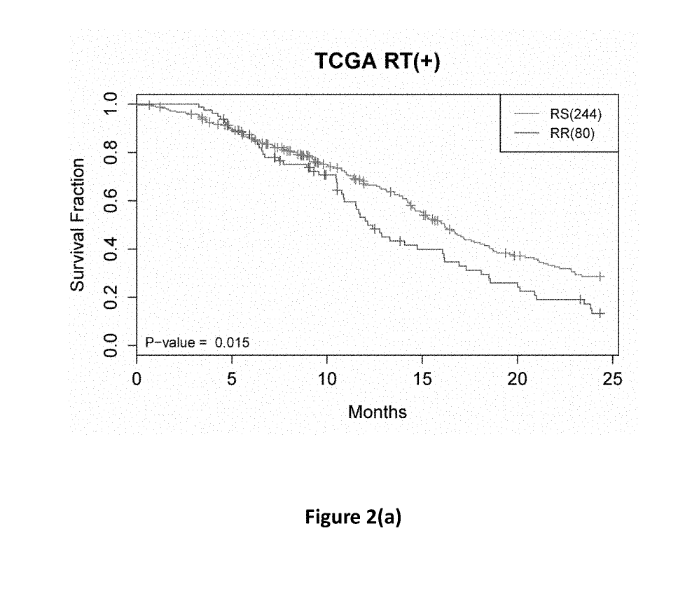

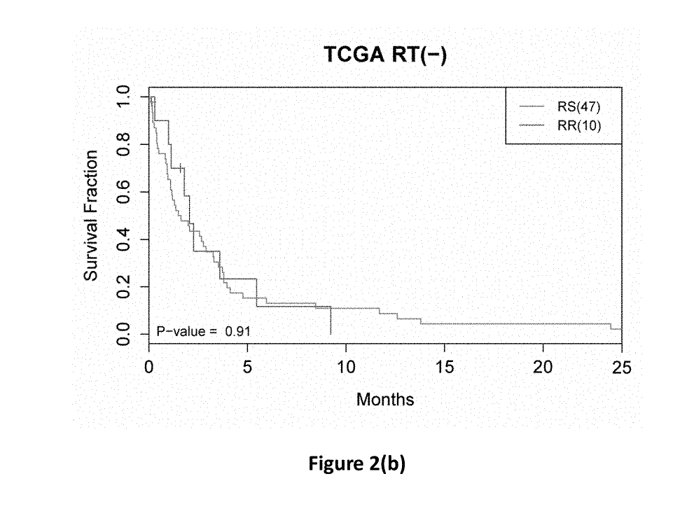

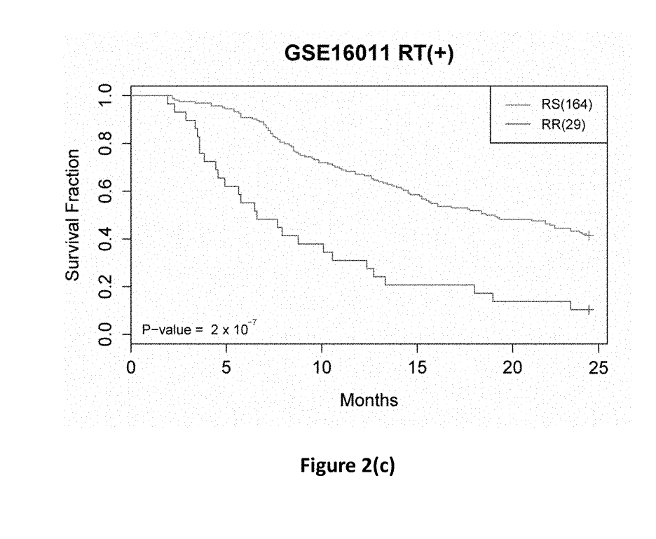

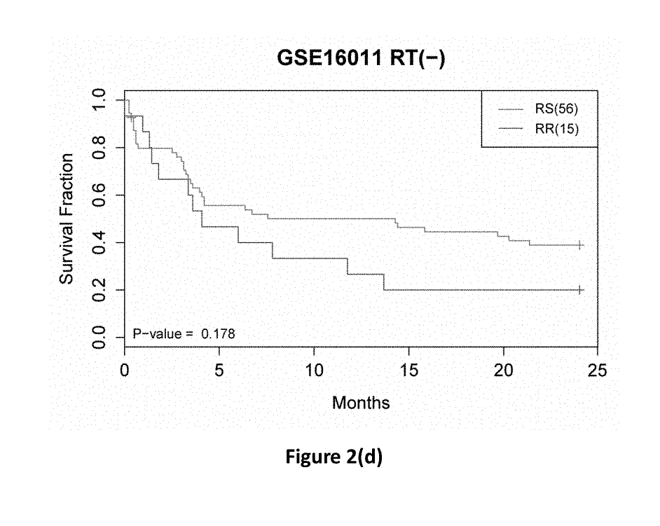

The developed prediction model was applied to 2 microarray datasets of GBM patients from GSE16011 and TCGA. Considering the high mortality rate of GBM, we focused on the overall survival rate within 2 years. Patients' data were excluded from the analyses if no information was provided about their status of radiotherapy and survival. The stratification resulted in 324 RT+ and 57 RT− patients in the GBM dataset from TCGA and 193 RT+ and 70 RT− patients in GSE16011. The developed prediction model was utilized to classify those patients into RS or RR groups, and log-rank tests were used to examine the differences in overall survival between them. To further evaluate whether the prediction model was associated with radiotherapy only, the RT+ and RT− patients were compared accordingly. Kaplan-Meier survival curves (as shown in FIGS. 2(a), 2(b), 2(c) and 2(d)) were produced and Cox hazard regression analyses were performed to assess the differences in and performance of the prediction model and other clinical parameters.

A multivariable Cox hazard regression model was performed to compare the prediction performance of the model with other clinical parameters, including age, grade, and Karnofsky Performance Score (KPS). As shown in Table 4, the prediction model remains an independent predictor after being adjusted for other confounding factors (P=0.00698 for TCGA and P=0.0277 for GSE16011). Therefore, these results demonstrate that the prediction model can effectively identify patients who will be responsive to radiotherapy and thus experience significantly better survival outcomes.

TABLE 4

| | | Hazard | | |
|---|---|---|---|---|
| | | ratio | SE | P-value |
| TCGA RT(+) | Model | 1.641 | 0.184 | 6.98E−03 |
| | Age | 1.033 | 0.007 | 4.02E−06 |
| | Chemotherapy | 0.513 | 0.297 | 2.45E−02 |
| | KPS | 0.992 | 0.006 | 2.14E−01 |
| TCGA RT(−) | Model | 0.836 | 0.506 | 7.24E−01 |
| | Age | 1.028 | 0.020 | 1.79E−01 |
| | Chemotherapy | 1.079 | 0.427 | 8.58E−01 |
| | KPS | 0.972 | 0.015 | 4.83E−02 |
| GSE16011 RT(+) | Model | 1.681 | 0.236 | 2.77E−02 |
| | Histological diagnosis | 0.615 | 0.146 | 8.75E−04 |
| | WHO grade | 2.227 | 0.191 | 2.74E−05 |
| | Gender | 0.932 | 0.199 | 7.22E−01 |
| | Age | 1.037 | 0.008 | 3.41E−06 |
| | KPS | 0.989 | 0.006 | 7.34E−02 |
| | Type of surgery | 1.179 | 0.090 | 6.77E−02 |
| | Chemotherapy | 0.788 | 0.343 | 4.87E−01 |
| GSE16011 RT(−) | Model | 0.604 | 0.417 | 2.26E−01 |
| | Histological diagnosis | 0.796 | 0.249 | 3.59E−01 |
| | WHO grade | 2.405 | 0.349 | 1.18E−02 |
| | Gender | 2.237 | 0.368 | 2.87E−02 |
| | Age | 1.007 | 0.016 | 6.83E−01 |
| | KPS | 0.961 | 0.010 | 2.71E−05 |
| | Type of surgery | 1.363 | 0.160 | 5.24E−02 |

As so far, the present invention is the first study to incorporate the expression levels of lncRNAs into a prediction model for radiosensitivity. Furthermore, a permutation test was performed in this study to assess whether it is superior to add lncRNAs into a prediction model for radiosensitivity.

In the development of a prediction model, a critical step is how to select important markers (predictors), especially when handling high-throughput data. A popular approach is to perform a stepwise regression using forward selection and/or backward elimination, and to evaluate the performance by the Akaike information criterion (AIC). However, such methods are time-consuming and inefficient, because the number of possible combinations of predictors is large. We tried such an approach in this study, but the predictors identified from the AIC model changed substantially upon the addition of only 1 or 2 pairs of genes and lncRNAs. The GA was used instead due to its low computational complexity and high efficiency in achieving good accuracy. In this study, the number of possible combinations of the original pool in this study (34 genes and 7 lncRNAs) is C₂₀⁴¹=269128937220, which is impossible to test in a reasonable amount of time. In addition, it is well-known that radiosensitivity differs greatly across different tissues. Therefore, we analyzed the NCI-60 cell lines to develop the prediction model because they were from distinct tissue types. One major advantage of using the GA in this situation is that it can identify the best combination of predictors (genes and lncRNAs) through random selection over many generations, taking the heterogeneity of radiosensitivity in different tissue types into consideration. The randomness in the initial step of GA is compensated for by the reproducibility of the consecutive generations. Although it is desirable to minimize the number of predictors in the GA model in order to minimize costs and simplify experimental procedures, the prediction performance of the GA was poor and unstable when the number of predictors selected was <20. Therefore, the number of predictors in the GA was set at 20 to achieve the highest possible prediction accuracy with the minimum number of predictors.

The present invention is to identify markers (predictors) based on NCI-60 cell lines and then to validate their performance in patient cohorts. Therefore, we developed the prediction model for radiosensitivity based on gene expression data. Furthermore, it is not difficult to get lymphocytes specimen from patients with glioblastoma; therefore peripheral blood samples are suggested as the source for future applications. Third, the prediction outcomes were dichotomized into RS and RR groups by SVM algorithm based on the SF2 parameter.

Accordingly, in the present invention, the expression levels of both genes and long non-coding RNAs (lncRNAs) were used to build such a prediction model. Analysis of variance and Tukey's honest significant difference tests (P<0.001) were utilized in immortalized B cells (GSE26835) to identify differentially expressed genes and lncRNAs after irradiation. A total of 41 genes and lncRNAs associated with radiation exposure were revealed by a network analysis algorithm. To develop a predictive model for radiosensitivity, the expression profiles of NCI-60 cell lines along, with their radiation parameters, were analyzed. A genetic algorithm was proposed to identify 20 markers, and the support vector machine algorithm was used to evaluate their prediction performance. The model was applied to 2 datasets of glioblastoma, The Cancer Genome Atlas and GSE16011, and significantly better survival was observed in patients with greater predicted radiosensitivity

While the invention has explained in relation to its preferred embodiments, it is well understand that various modifications thereof will become apparent to those skilled in the art upon reading the specification. Therefore, the invention disclosed herein intended to cover such modifications as fall within the scope of the appended claims.

What is claimed is:

1. A method for identifying radiation induced genes and long non-coding RNAs, comprising:
(1). Providing expression values of genes and long non-coding RNAs;
(2). Executing weighted gene correlation network analysis (WGCNA) by a computer system to calculate Pearson correlation coefficients of pairs of the genes and long non-coding RNAs based on the expression values of the genes and long non-coding RNAs; and
(3). Performing a screening step by the computer system to identify radiation induced genes and long non-coding RNAs based on the Pearson correlation coefficients of the pairs of the genes and long non-coding RNAs with a value more than 0.75.

2. The method of claim 1, wherein the expression values of genes and long non-coding RNAs are measured in vitro from immortalized B cells after irradiation by a method comprises microarray.

3. The method of claim 1, wherein the radiation induced genes are selected from the group consisting of DDX3Y, EIF1AY, RPS4Y1, USP9Y, KDM5D, TP53I3, RHOC, ASTN2, GAMT, EPS8L2, ACTA2, E2F5, MR1, UTY, TRAPPC6A, FHL2, ANXA4, GLS2, CEACAM1, ETHE1, TSPAN31, PYCARD, CDK2, JUP///KRT17, ATP6V1D, PROCR, ETFDH, ALDH6A1 and RTN1.

4. The method of claim 1, wherein the radiation induced long non-coding RNAs are selected from the group consisting of TTTY15, TP53TG1, LOC100653079, LOC100653017 and LOC100506948.

5. The method of claim 3, wherein the radiation induced genes selected from the group consisting of DDX3Y, EIF1AY, RPS4Y1, USP9Y, RHOC, EPS8L2, ACTA2, MR1, TRAPPC6A, ANXA4, ETHE1, PYCARD, JUP///KRT17, ETFDH, ALDH6A1 and RTN1 are applied as markers for patients with glioblastoma.

6. The method of claim 4, wherein the radiation induced long non-coding RNAs selected from the group consisting of TP53TG1, LOC100653017 and LOC100506948 are applied as markers for patients with glioblastoma.

7. A method for predicting radiotherapy response of glioblastoma in patients with glioblastoma, comprising:
(1). determining expression values of markers in a test sample from the patients with glioblastoma to obtain a test dataset of expression values of the markers, wherein the markers are selected from the group consisting of DDX3Y, EIF1AY, RPS4Y1, USP9Y, RHOC, EPS8L2, ACTA2, MR1, TRAPPC6A, ANXA4, ETHE1, PYCARD, JUP///KRT17, ETFDH, ALDH6A1, RTN1, TP53TG1, LOC100653017 and LOC100506948;
(2). comparing the test dataset of expression values of the markers to a radiosensitive sample and to a radioresistant sample;
(3). classifying whether a test dataset of expression values of the markers is significantly within the radiosensitive sample or within the radioresistant sample to assess whether the test sample is radiosensitive or radioresistant;
(4). determining the patients with glioblastoma as in a radiosensitive group if the test sample is radiosensitive, or determining the patients with glioblastoma as in a radioresistant group if the test sample is radioresistant; and
(5). predicting radiotherapy response of glioblastoma in the patients with glioblastoma based on whether the radiosensitive group or radioresistant group is treated with radiotherapy.

8. The method of claim 7, being applied to a prognosis analysis of glioblastoma in the patients with glioblastoma treated with radiotherapy.

9. The method of claim 7, wherein the expression values of the markers are measured in vitro by a method comprises microarray and real-time polymerase chain reaction (RT-PCR).

10. The method of claim 7, wherein the test sample comprises lymphocytes sample and peripheral blood sample.

11. The method of claim 7, wherein the radiosensitive sample and radioresistant sample are determined by a decision value calculated from the expression values of the markers in NCI-60 cell lines by support vector machine, when the decision value is negative, the radiosensitive sample is defined; and when the decision value is positive, the radioresistant sample is defined.

12. The method of claim 7, wherein classifying whether the test dataset of expression values of the markers is significantly within the radiosensitive sample or within the radioresistant sample comprises use of a predictive algorithm.

13. The method of claim 12, wherein the predictive algorithm is a support vector machine.

14. The method of claim 7, wherein the steps of (2), (3), (4) and (5) are performed by a computer system.
